(19) United States
(12) Patent Application Publication
(10) Pub. No.: US 2008/0305159 A1
(43) Pub. Date: Dec. 11, 2008

(54) Ibuprofen-Hydrocodone-Antihistamine Composition

(75) Inventor: Pieter J. Groenewoud, Powder Springs, GA (US)

Correspondence Address: BRYAN W. BOCKHOP, ESQ.;BOCKHOP & ASSOCIATES, LLC, 2375 MOSSY BRANCH DR., SNELLVILLE, GA, 30078, US

(73) Assignee: MIKART, INC., Atlanta, GA (US)

(21) Appl. No.: 12/134,612

(22) Filed: Jun. 6, 2008

Related U.S. Application Data
(60) Provisional application No. 60/942,372, filed on Jun. 6, 2007.

(51) Int. Cl. A61K 9/66; A61K 31/4355; A61P 43/00

(52) U.S. Cl. 424/453; 514/282

(57) ABSTRACT

A pharmaceutical composition for use in relieving cold and cough symptoms in a mammal includes ibuprofen, hydrocodone bitartrate and an antihistamine (or a decongestant). The ibuprofen is in a therapeutically effective amount sufficient to act as an analgesic and an anti-inflammatory. The hydrocodone bitartrate is in a therapeutically effective amount sufficient to act as an antitussive and an analgesic. The antihistamine is in a therapeutically effective amount to act as an antihistamine. A fatty excipient may be added to the active ingredients to facilitate encapsulation. Multiple layers of the fatty excipient may be employed in varying release formulations and to facilitate identification of the composition.

### CROSS-REFERENCE TO RELATED APPLICATION(S)

This application claims the benefit of U.S. Provisional Patent Application Ser. No. 60/942,372, filed Jun. 6, 2007, the entirety of which is hereby incorporated herein by reference.

### BACKGROUND OF THE INVENTION

1. Field of the Invention

The present invention relates to pharmaceutical compositions and, more specifically, to a pharmaceutical composition directed to treatment of cold and cough symptoms.

2. Description of the Prior Art

Most people experience upper respiratory tract congestion due to infections or allergies. Such congestion usually results in coughing, irritation and soreness in the afflicted person's throat.

There are a variety of remedies used to treat upper respiratory tract congestion. These include “home” remedies, over-the-counter remedies and prescribed remedies. Examples of home remedies include warm drinks and application of eucalyptus ointments. These remedies may be an effective treatment for mild congestion and may supplement other remedies when the congestion is more severe. Examples of over-the-counter remedies, which are also used to treat mild congestion, include analgesics (such as aspirin and ibuprofen), antihistamines (such as loratadine, chlorpheniramine and dexchlorpheniramine), decongestants (such as pseudo ephedrine) and antitussives (such as dextromethorphan). Over-the-counter remedies are sometimes formulated together. However, afflicted people often supplement over-the-counter remedies with other remedies and such combinations may be less than optimal. Also, recent studies have indicated that many over-the-counter remedies are no more effective than placebos (see, e.g., Schroeder and Fahey, “Systematic review of randomised controlled trials of over the counter cough medicines for acute cough in adults” British Medical Journal 324: 329-331 (2002)).

Prescribed remedies often include narcotics formulated as antitussives. Narcotics, while effective as antitussives, may be subject to abuse. They also can lead to a sense of disorientation.

Narcotics have been combined with other active ingredients (such as some of the ingredients discussed above) to treat a wider number of symptoms. For example, it is know to combine codeine with acetaminophen or ibuprofen. It is also known to combine narcotics with antihistamines and decongestants (see, e.g., U.S. Pat. No. 6,979,689, issued to Gonzales et al.). Combining narcotics with other ingredients can treat several symptoms with a single dose.

While several combinations exist, none of the existing formulations treat all of the symptoms relating to congestion. Because of this, patients are sometimes given increased doses of one ingredient in an attempt to treat the symptoms that are best treated by another ingredient.

Therefore, there is a need for a well balanced combination of ingredients directed to treating all of the symptoms associated with congestion.

### SUMMARY OF THE INVENTION

In one aspect, the invention is a pharmaceutical composition for use in relieving cold and cough symptoms in a mammal, that includes ibuprofen, hydrocodone bitartrate and an antihistamine. The ibuprofen, or a salt thereof, is in a therapeutically effective amount sufficient to act as an analgesic and an anti-inflammatory. The hydrocodone bitartrate is in a therapeutically effective amount sufficient to act as an antitussive and an analgesic. The antihistamine is in a therapeutically effective amount to act as an antihistamine.

In another aspect, the invention is a pharmaceutical composition for use in relieving cold and cough symptoms in a mammal that includes ibuprofen, hydrocodone bitartrate and a decongestant. The ibuprofen, or a salt thereof, is in a therapeutically effective amount sufficient to act as an analgesic and an anti-inflammatory. The hydrocodone bitartrate is in a therapeutically effective amount sufficient to act as an antitussive and an analgesic. The decongestant is in a therapeutically effective amount to act as an decongestant.

### BRIEF DESCRIPTION OF THE FIGURES OF THE DRAWINGS

FIGS. 1A-1D are schematic drawings showing a multi-layered capsule.

FIG. 2A-2B are schematic drawings showing a sealable capsule that is configured to contain a liquid.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

In one embodiment, the invention is a pharmaceutical composition for use in relieving cold and cough symptoms in a mammal. The composition includes, as active ingredients, ibuprofen, or a salt thereof, in a therapeutically effective amount sufficient to act as an analgesic and an anti-inflammatory, hydrocodone bitartrate in a therapeutically effective amount sufficient to act as an antitussive and an analgesic, and an antihistamine in a therapeutically effective amount to act as an antihistamine.

The antihistamine could include, for example, dexchlorpheniramine maleate or chlorpheniramine maleate in a night time formulation, or loratadine in a day time formulation. Racemic ibuprofen would typically be employed in the composition.

Typically, the composition would be made by mixing the active ingredients in powder form with any necessary excipients and then granulated using well known pharmaceutical mixing and granulation methods. The granules could then be placed in capsules or compacted into tablets.

In one embodiment, ibuprofen, hydrocodone and antihistamine may suspended in a fatty excipient that has a liquid phase at a temperature greater than a melting point and that has a solid phase at a temperature not greater than the melting point. When a polyglycolized glyceride is employed, the a polyglycolized glyceride is melted into liquid form, the active ingredients and any necessary excipients are added, the resulting liquid is then poured into the delivery vehicle (such as a capsule) and then the liquid is allowed to cool into a solid.

The fatty excipient could include both an immediate release formulation and an extended release formulation. Such a formulation would offer the advantage of providing the patient with immediate relief and subsequent sustained relief from the cold symptoms experienced by the patient.

It is believed that the combination of ibuprofen, hydrocodone and antihistamine act synergistically to relieve cough and cold symptoms. The ibuprofen reduces inflammation, thereby reducing the irritation level in the tissues that produce excess mucous. The antihistamine also reduces the amount of excess mucous produced in the tissues. The ibuprofen and the hydrocodone both act as an analgesic, which reduces the discomfort experienced by the patient. Thus, the combination of the anti-inflammatory effect of the ibuprofen, the antihistamine effect of the antihistamine and the analgesic effects of the ibuprofen and the hydrocodone reduces the antitussive level required to achieve a desired comfort level. Typically, the dosage would include the following ingredients: 100 mg to 400 mg Ibuprofen; 2.5 mg to 10 mg hydrocodone bitartrate; and 2 mg to 24 mg chlorpheniramine maleate. One representative dosage includes 200 mg Ibuprofen, 5 mg hydrocodone bitartrate and 2 mg chlorpheniramine maleate.

In an alternate embodiment, the invention is a pharmaceutical composition for use in relieving cold and cough symptoms in a mammal. The composition includes, as active ingredients, ibuprofen, or a salt thereof, in a therapeutically effective amount sufficient to act as an analgesic and an anti-inflammatory, hydrocodone bitartrate in a therapeutically effective amount sufficient to act as an antitussive and an analgesic, and a decongestant in a therapeutically effective amount to act as a decongestant. In one embodiment, the decongestant includes pseudo-ephedrine. Typically, the dosage would include the following ingredients: 100 mg to 400 mg Ibuprofen; 2.5 mg to 10 mg hydrocodone bitartrate; and 30 mg to 120 mg pseudo-ephedrine. One representative dosage includes 200 mg Ibuprofen, 5 mg hydrocodone bitartrate and 60 mg of pseudo-ephedrine.

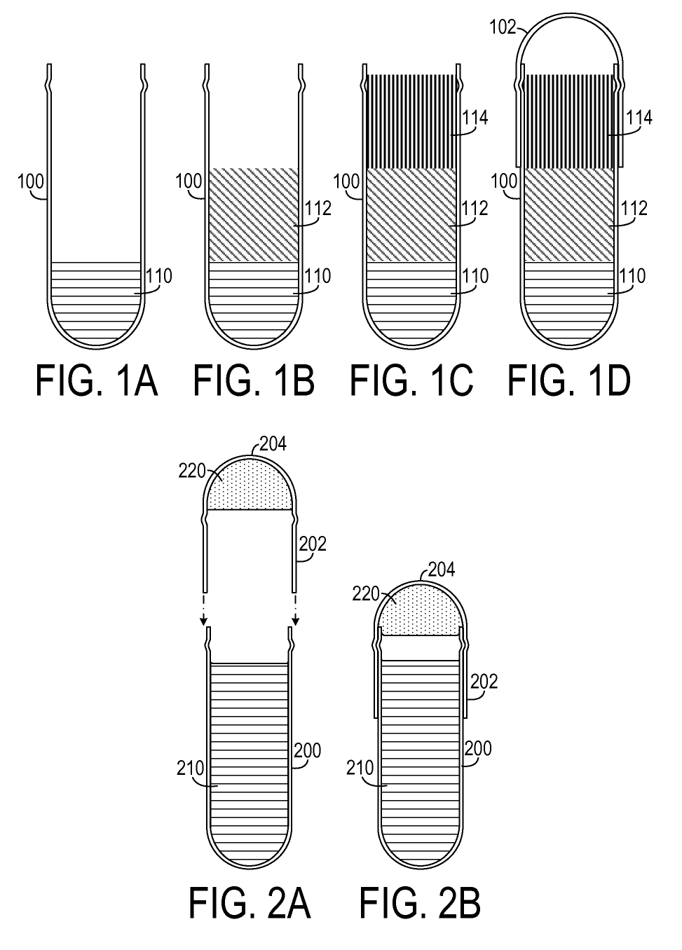

In one embodiment, as shown in FIGS. 1A-1D, a first composition is dissolved or suspended in a fatty excipient (e.g., a polyglycolized glyceride, such as Gelucire®) that has been melted (for example, one formulation of Gelucire® has a melting point of 43° C.) into the liquid phase and then poured into a capsule 100. The first composition is allowed to cool until it is in a solid phase, thereby forming a first layer 110. Then a second composition is poured into the capsule 100 while in the liquid phase and is also allowed to cool to form a solid second layer 112. A third composition may be poured into the capsule 100 on top of the second layer 112 and allowed to cool to form a solid third layer 114. For example, the first layer 110 could include the hydrocodone, the second layer 112 could include the ibuprofen and the third layer 114 could include the antihistamine. In another example, each layer 110, 112 and 114 would include all three ingredients, but the first layer 110 would include an immediate release formulation, the second layer 112 would include a first delayed release formulation that would become substantially dissolved near when the immediate release formulation begins to loose effect, and the third layer would include a second delayed release formulation that would become substantially dissolved near when the first delayed release formulation begins to loose effect. This approach might be particularly useful in all-night formulations.

In one embodiment, a different color dye may be added to each layer so that the layers color code the capsule, indicating the contents to the user through visual inspection. In one example, a dye of a first color may be added to an immediate release formulation and a dye of a different second color may be added to the extended release formulation. The immediate release formulation would disposed in a capsule in a first layer and the extended release formulation would be disposed in the capsule in a second layer disposed adjacent and on top of the first layer. This two-layered formulation would allow for easy identification by the user.

This multi-colored layered concept may be applied to other formulations as well. The different colors of the layers (which could number more than two) could signify different active ingredients, or different release timings. Such color coding has the advantage of allowing the user to identify what type of capsule is being taken and makes the capsule more inviting to patients.

One embodiment of the invention is a pharmaceutical delivery vehicle that facilitates placement of a liquid active ingredient into a conventional capsule. A liquid active ingredient is mixed into a polyglycolized glyceride excipient while in the liquid phase and the resulting composition is poured into a capsule. The composition is allowed to cool to a solid phase and then the capsule is capped.

In another embodiment, as shown in FIGS. 2A and 2B, a pharmaceutical capsule for delivery of a liquid ingredient 210 includes a body member 200 into which is poured the liquid ingredient 210. A cap member 202 that is configured to cap the body member includes an end portion 204. A waxy or fatty excipient 220 is placed in the end portion 204. The waxy or fatty excipient 220 has a thickness so that when the cap member 202 is applied to the body member 200, the open top portion of the body member 200 will be sealed by the fatty or waxy excipient 220.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A pharmaceutical composition for use in relieving cold and cough symptoms in a mammal, comprising:
a. ibuprofen, or a salt thereof, in a therapeutically effective amount sufficient to act as an analgesic and an anti-inflammatory;
b. hydrocodone bitartrate in a therapeutically effective amount sufficient to act as an antitussive and an analgesic; and
c. an antihistamine in a therapeutically effective amount to act as an antihistamine.

2. The pharmaceutical composition of claim 1, wherein the antihistamine consists of chlorpheniramine maleate and wherein active ingredients of the composition consist of:
a. 100 mg to 400 mg Ibuprofen;
b. 2.5 mg to 10 mg hydrocodone bitartrate; and
c. 2 mg to 24 mg chlorpheniramine maleate.

3. The pharmaceutical composition of claim 1, wherein the antihistamine is selected from a group consisting of: chlorpheniramine maleate; dexchlorpheniramine maleate, loratadine, and combinations thereof.

4. The pharmaceutical composition of claim 1, wherein the ibuprofen comprises racemic ibuprofen.

5. The pharmaceutical composition of claim 1, wherein the ibuprofen, the hydrocodone and the antihistamine are suspended in a fatty excipient that has a liquid phase at a temperature greater than a melting point and that has a solid phase at a temperature not greater than the melting point.

6. The pharmaceutical composition of claim 5, wherein the melting point comprises 43° C.

7. The pharmaceutical composition of claim 5, wherein the fatty excipient comprises a polyglycolized glyceride.

8. The pharmaceutical composition of claim 5, wherein the fatty excipient comprises:
a. an immediate release formulation; and
b. an extended release formulation.

9. The pharmaceutical composition of claim 8, wherein the immediate release formulation is suspended in a first layer including a polyglycolized glyceride excipient in which a dye of a first color is included therein, and wherein the extended release formulation is suspended in a second layer, disposed adjacent to the first layer, the second layer including a polyglycolized glyceride excipient in which a dye of a second color, different from the first color, is included therein.

10. The pharmaceutical composition of claim 1, wherein the ibuprofen, the hydrocodone and the antihistamine are suspended in a liquid to form a suspension and wherein the suspension is poured into a capsule that comprises:
a. a body member configured to receive the liquid ingredient therein, the body member including an open top portion;
b. a cap member configured to cap the body member, the cap member including an end portion; and
c. a waxy excipient disposed in the end portion and having a thickness so that when the cap member is applied to the body member, the open top portion will be sealed by the waxy excipient.

11. A pharmaceutical composition for use in relieving cold and cough symptoms in a mammal, comprising:
a. ibuprofen, or a salt thereof, in a therapeutically effective amount sufficient to act as an analgesic and an anti-inflammatory;
b. hydrocodone bitartrate in a therapeutically effective amount sufficient to act as an antitussive and an analgesic; and
c. a decongestant in a therapeutically effective amount to act as an decongestant.

12. The pharmaceutical composition of claim 11, wherein active ingredients of the composition consist of:
a. 100 mg to 400 mg Ibuprofen;
b. 2.5 mg to 10 mg hydrocodone bitartrate; and
c. 30 mg to 120 mg pseudo-ephedrine.

13. The pharmaceutical composition of claim 11, wherein the decongestant consists of pseudo-ephedrine.

14. The pharmaceutical composition of claim 11, wherein the ibuprofen comprises racemic ibuprofen.

15. The pharmaceutical composition of claim 11, wherein the ibuprofen, the hydrocodone and the decongestant are suspended in a fatty excipient that has a liquid phase at a temperature greater than a melting point and that has a solid phase at a temperature not greater than the melting point.

16. The pharmaceutical composition of claim 15, wherein the melting point comprises 43° C.

17. The pharmaceutical composition of claim 15, wherein the fatty excipient comprises a polyglycolized glyceride.

18. The pharmaceutical composition of claim 15, wherein the fatty excipient comprises:
a. an immediate release formulation; and
b. an extended release formulation.

19. The pharmaceutical composition of claim 15, wherein the immediate release formulation is suspended in a first layer including a polyglycolized glyceride excipient in which a dye of a first color is included therein, and wherein the extended release formulation is suspended in a second layer, disposed adjacent to the first layer, the second layer including a polyglycolized glyceride excipient in which a dye of a second color, different from the first color, is included therein.

20. The pharmaceutical composition of claim 19, wherein the ibuprofen, the hydrocodone and the decongestant are suspended in a liquid to form a suspension and wherein the suspension is poured into a capsule that comprises:
a. a body member configured to receive the liquid ingredient therein, the body member including an open top portion;
b. a cap member configured to cap the body member, the cap member including an end portion; and
c. a waxy excipient disposed in the end portion and having a thickness so that when the cap member is applied to the body member, the open top portion will be sealed by the waxy excipient.
